(19) United States
(12) Patent Application Publication
(10) Pub. No.: US 2022/0287341 A1
(43) Pub. Date: Sep. 15, 2022

(54) EDIBLE CHEW FOR A HUMAN CHILD AND METHODS OF MAKING AND USING THE EDIBLE CHEW

(71) Applicant: SOCIETE DES PRODUITS NESTLE S.A., Vevey (CH)

(72) Inventors: STEPHANIE CAROLE HENRIETTE DARU, Vevey (CH); NORA SCHNEIDER, Vevey (CH); ZOOBIA TAHIR, Vevey (CH); YANNICK MAHE, Vevey (CH); LAURENT JEAN-JACQUES SISIAK, Vevey (CH); SARAH ELIZABETH SMITH-SIMPSON, Vevey (CH)

(21) Appl. No.: 17/754,175

(22) PCT Filed: Apr. 10, 2020
(86) PCT No.: PCT/IB2020/053451

§ 371 Date: Mar. 25, 2022

Related U.S. Application Data
(63) Continuation of application No. 16/691,997, filed on Nov. 22, 2019.
(60) Provisional application No. 62/906,773, filed on Sep. 27, 2019.

(51) Int. Cl. A23L 7/122; A23L 33/00; A23L 29/30; A23L 33/15; A23P 30/20; A23L 33/18; A23L 33/135; A23L 33/105; A23L 33/175

(52) U.S. Cl. CPC A23L 7/122; A23L 33/40; A23L 29/37; A23L 33/15; A23P 30/20; A23L 33/18; A23L 33/135; A23L 33/105; A23L 33/175

(57) ABSTRACT

An edible chew is formulated for oral consumption by a human child and includes an expanded pre-gelatinized cereal flour matrix. A method of (i) providing teething support to a human child who is teething and/or (ii) soothing, calming and/or delivering an active ingredient to a human child can include administering the edible chew to the human child. Methods of making the edible chew are provided, for example by modified extrusion; and also a system including the edible chew and further including a holder configured to attach to the edible chew; a product including a packaging and further including the edible chew; and a product including a container housing a plurality of the edible chews, and the plurality of edible chews are individually packaged.

### CROSS REFERENCE TO RELATED APPLICATIONS

This application claims priority to U.S. Provisional Application No. 62/906,773 filed Sep. 27, 2019 and U.S. Non-Provisional application Ser. No. 16/691,997 filed Nov. 22, 2019, the entire contents of which are incorporated herein by reference.

### BACKGROUND

The present disclosure relates generally to health and nutrition of a human child. More specifically, the present disclosure relates to an edible chew formulated for oral administration to the human child, a product comprising packaging and the edible chew therein, a method of making the edible chew, and a method of using the edible chew.

The teething period is a difficult time in the life of a baby, and most of the time, parents do not know what to do to help the baby with the pain. Different attempted solutions are available commercially, but they act either on biting needs or the pain and do not address both needs. For example, a numbing gel is hard to apply and does not last very long. Paracetamol/acetaminophen is a drug and should not be administered frequently to a baby. A biscuit, wafer, or rusk is too soft, dissolves too fast, and has no nutritional value. Teething biscuits like Bickiepegs are small, hard, and do not dissolve and can pose a choking hazard and are hard for a baby to hold. A plastic toy can help for biting but does not relieve pain, and further more the plastic toy can leach a toxic chemical or break into pieces of plastic.

Moreover, edible chews for dogs are known, but such edible chews are not suitable for a human child. For example, the function of these known edible chews for dogs is cleaning teeth, whereas an edible chew for a human baby should massage the gums of the human child. Furthermore, the known edible chews for dogs are too big and too heavy to be held by the hand of a six-month old human baby. Moreover, the known edible chews for dogs have a formulation containing ingredients which are not adapted to and allowed for baby consumption in a teether.

### SUMMARY

In a non-limiting embodiment, the present disclosure generally provides an edible chew that can have a long stick shape that can have a diameter allowing toddlers and also infants as young as six months old (and even younger in some embodiments) to hold the edible chew and place the edible chew in their mouth. The edible chew can have a thin teether wall surrounding a hollow center. The edible chew can have a teether texture that is spongy and soft in the mouth, slow to dissolve, and dissolves in small pieces instead of chunks. In an embodiment, the edible chew dissolves in greater than 5 minutes. In a further embodiment, the edible chew dissolves in greater than 10 minutes. In yet a further embodiment, the edible chew dissolves in greater than 15 minutes.

Methods of making the edible chew and methods of using the edible chew are also disclosed herein. For example, the edible chew can be provided as a teething support to a child in need thereof (e.g., an infant or toddler). As another example, the edible chew can be provided to a child (e.g., an infant or toddler) to sooth the child, calm the child, and/or deliver an active ingredient to the child.

In an embodiment, the present disclosure provides an edible chew formulated for oral consumption by a human child, the edible chew comprising an expanded pre-gelatinized cereal flour matrix.

In another embodiment, the present disclosure provides a method of (i) providing teething support to a human child who is teething and/or (ii) soothing, calming and/or delivering an active ingredient to a human child, the method comprising orally administering the edible chew to the human child.

In yet other embodiments, the present disclosure provides a system comprising the edible chew and further comprising a holder configured to attach to the edible chew; a product comprising a packaging and further comprising the edible chew therein; and a product comprising a container housing a plurality of the edible chews, wherein the plurality of edible chews are individually packaged.

An advantage provided by one or more embodiments disclosed herein is an edible chew made entirely from real food ingredients without plastic or medicine.

Another advantage provided by one or more embodiments disclosed herein is to massage teething gums of a baby.

Yet another advantage provided by one or more embodiments disclosed herein an edible chew with appealing taste for a child, for example one or more of cereal, a fruit such as banana, a vegetable such as spinach or beet juice, a herb or a spice.

Another advantage provided by one or more embodiments disclosed herein is an edible chew with a hollow design that reduces a risk of choking or prevents choking for a child eating the edible chew.

Yet another advantage provided by one or more embodiments disclosed herein is an edible chew with a spongy, flexible texture.

Another advantage provided by one or more embodiments disclosed herein is an edible product that soothes a child's gums as they chew the product.

Yet another advantage provided by one or more embodiments disclosed herein is an edible chew that is gum friendly. As used herein, gum friendly is intended to mean a baby chew that is: noncariogenic; promotes health balance of oral microflora; soothing to the gums and oral surfaces; stimulating to the gums and oral surfaces; non-irritating to the gums and other oral surfaces; cleaning to the gums and other oral surfaces; massaging to the gums and other oral surfaces; supporting tooth eruption; designed for gnawing, chewing, and/or bruxism; or combinations therefrom.

Another advantage provided by one or more embodiments disclosed herein is an edible product that dissolves slowly to provide long-lasting chewing comfort to a child. In an embodiment, the edible chew dissolves in greater than 5 minutes. In a further embodiment, the edible chew dissolves in greater than 10 minutes. In yet a further embodiment, the edible chew dissolves in greater than 15 minutes. In an embodiment, the edible chew does not dissolve.

Yet another advantage provided by one or more embodiments disclosed herein is an edible chew that has a size and shape such that the edible chew is easy for a child to grip and hold in one hand or two hands, easy for a child to grip and hold in the mouth, and also fun for the child to play with.

Another advantage of provided by one or more embodiments disclosed herein is an edible chew providing calcium for healthy teeth of a child.

Yet another advantage provided by one or more embodiments disclosed herein is to relieve helplessness, anxiety and/or frustration of a parent having a child in pain caused by teething gums.

Another advantage provided by one or more embodiments disclosed herein is an edible chew that lasts longer than other teething snacks.

Another advantage provided by one or more embodiments disclosed herein is an edible chew that is designed for healthy teething.

Yet another advantage of provided by one or more embodiments disclosed herein is an edible chew that does not adhere to gums or teeth of a child eating the edible chew.

Another advantage provided by one or more embodiments disclosed herein is an edible chew that is safe and gentle for emerging teeth of a child.

Yet another advantage provided by one or more embodiments disclosed herein is an edible chew that occupies the interest of a child eating the edible chew, for example more than ten minutes and even twenty to thirty minutes.

Another advantage provided by one or more embodiments disclosed herein is an edible chew that has a texture that encourages chewing by a child and helps the child practice chewing.

Yet another advantage provided by one or more embodiments disclosed herein is an edible chew sized and shaped for a child to practice eye-hand-mouth coordination.

Another advantage provided by one or more embodiments disclosed herein is an edible chew sized and shaped to help a child develop sense of touch and coordination.

Yet another advantage provided by one or more embodiments disclosed herein is an edible chew provided with a holder that easily attaches to the edible chew and a child eating the edible chew to ensure the edible chew maintains proximity to the child.

Another advantage provided by one or more embodiments disclosed herein is an edible chew that can be chilled or frozen, for example to provide cooling to gums of a teething human child consuming the chilled or frozen edible chew.

Yet another advantage provided by one or more embodiments disclosed herein is an edible chew that is visually appealing to both the child and their mother, for example with one or more of a hollow design, a round edge that provides safety, and a bright natural color such as yellow.

Another advantage provided by one or more embodiments disclosed herein is an edible chew having a natural taste without strong flavor.

Yet another advantage provided by one or more embodiments disclosed herein is an edible chew that has a texture with elasticity that alleviates teething pain of a child eating the edible chew.

Another advantage provided by one or more embodiments disclosed herein is an edible chew that has air bubbles throughout the edible chew to establish a spongy texture.

Yet another advantage provided by one or more embodiments disclosed herein is an edible chew that provides an active ingredient, for example one or more of a vitamin, a mineral, chamomile, lavender, a sugar alcohol/polyol other than glycerol, a cooling sensate such as menthol, a micronutrient, a nutraceutical, a prebiotic, a probiotic, a protein, an amino acid, an essential oil, a homeopathic ingredient, or a pharmaceutical.

Another advantage provided by one or more embodiments disclosed herein is an edible chew that can be used for soothing, calming and/or relief of discomfort, pain or stress for a child.

Yet another advantage provided by one or more embodiments disclosed herein is an edible chew that treats or prevents inflammation, improves oral microbiota, provides teething support (e.g., for erupting teeth), provides stress relief, improves mood, stimulates brain activity, provides oral-motor benefits such as assisting language development, enhances motor development such as hand-eye coordination, and/or has less hygiene issues relative to other multi-use products.

Another advantage provided by one or more embodiments disclosed herein is an edible chew that can be eaten by a child as a snack.

Yet another advantage provided by one or more embodiments disclosed herein is an edible chew that is less hazardous than known teethers.

Another advantage provided by one or more embodiments disclosed herein is an edible chew that has bright colors and/or varied colors to attract a child.

Yet another advantage provided by one or more embodiments disclosed herein is an edible chew that can be one or more of organic, gluten-free and nut-free.

Another advantage provided by one or more embodiments disclosed herein is an edible chew that includes a potential food allergen, such as nuts, for early tolerance introduction of a child.

Yet another advantage provided by one or more embodiments disclosed herein is an edible chew that has a nutritious paste filling.

Another advantage provided by one or more embodiments disclosed herein is an edible chew that maintains its taste throughout usage.

Yet another advantage provided by one or more embodiments disclosed herein is an edible chew that can be used as a teething tool for stimulating gums of a child, while releasing one or more nutrients.

Another advantage provided by one or more embodiments disclosed herein is an edible chew having an ergonomic shape.

Yet another advantage provided by one or more embodiments disclosed herein is a reusable holder or reusable stick for an edible chew that can house the last piece of the edible chew to prevent a child eating the edible chew from eating the last piece of the edible chew.

Another advantage provided by one or more embodiments disclosed herein is an edible chew that can be used to treat or prevent one or more of gastrointestinal (GI) distress, colic, or sore throat.

Yet another advantage provided by one or more embodiments disclosed herein is an edible chew that has no preservatives, no pesticides and no heavy metals.

Another advantage provided by one or more embodiments disclosed herein is an edible chew that can be made by modified extrusion.

Yet another advantage provided by one or more embodiments disclosed herein is an edible chew that has a mark thereon to identify an end portion that should not be eaten by the child.

Another advantage provided by one or more embodiments disclosed herein is an edible chew that is not slippery.

Yet another advantage provided by one or more embodiments disclosed herein is an edible chew that has no glycerol and instead contains a natural plasticizer component such as one or more of glucose syrup, apple juice concentrate, lactose or another short chain carbohydrate.

Another advantage provided by one or more embodiments disclosed herein is an edible chew that has a validated safety profile.

Yet another advantage provided by one or more embodiments disclosed herein is an edible chew that achieves all three of physical safety, developmentally appropriate, and assists behavioral development, for a child.

Another advantage provided by one or more embodiments disclosed herein is to address a teething child's desire to chew.

Yet another advantage provided by one or more embodiments disclosed herein is an edible chew made entirely from natural and organic ingredients.

Another advantage provided by one or more embodiments disclosed herein is an edible chew that is safe to provide to a baby even without constant supervision.

Yet another advantage provided by one or more embodiments disclosed herein is an edible chew that can have almost any density suitable for an edible product, for example a low density or a high density.

Additional features and advantages are described herein, and will be apparent from the following Detailed Description and the figures.

### BRIEF DESCRIPTION OF THE FIGURES

FIGS. 1 (1A, 1B, 1C, and 1D) illustrates a scientific developmental feeding model for a human child.

FIG. 2 illustrates a plurality of edible chews in accordance with an embodiment of the present disclosure.

FIGS. 3A and 3B illustrate a packaged edible chew in accordance with an embodiment of the present disclosure.

FIGS. 4A, 4B and 4C illustrate use of an edible chew with holders in accordance with embodiments of the present disclosure (Step 1—human adult has holder, Step 2—human adult positions the edible chew in the holder and/or on the holder, Step 3—human child eats top portion of edible chew while holder retains bottom portion of edible chew, Step 4—human adult takes holder from child with bottom portion of edible chew retained by holder and Step 5—bottom portion of edible chew removed from holder and disposed, preferably without disposing holder.

FIG. 5A illustrates video observation data showing that a majority of children aged 6-15 months exhibited feeding skills needed to successfully eat an edible chew in accordance with an embodiment of the present disclosure.

FIG. 5B illustrates video observation data showing that challenging behaviors were observed in a limited number of children aged 6-15 months, with no choking behaviors observed, from an edible chew in accordance with an embodiment of the present disclosure.

FIGS. 6A, 6B and 6C illustrate example formulations of an edible chew in accordance with an embodiment of the present disclosure.

FIG. 7 illustrates the study design of Example 3 disclosed herein.

FIG. 8 is data from Example 3 disclosed herein, regarding score of happiness and calmness, before and after teether exposure and averaged across babies and days of measurements. Bars=+/−SE; P(T<=t) one-tail t-test.

FIG. 9 is data from Example 3 disclosed herein, regarding score of stress and crankiness, before and after teether exposure and averaged across babies and days of measurements. Bars=+/−SE; P(T<=t) one-tail t-test.

FIG. 10 is data from Example 3 disclosed herein, regarding heart rate value before, during and after teether exposure and averaged across babies and days of measurements. Bars=+/−SE; P(T<=t) one-tail t-test.

### DETAILED DESCRIPTION

Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all amounts and all ratios are by weight unless expressed otherwise. When reference is made to the pH, values correspond to pH measured at 25° C. with standard equipment.

As used herein, “about,” “approximately,” “essentially” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an active ingredient” or “the active ingredient” includes a plurality of such “active ingredients.” The term “and/or” used in the context of “X and/or Y” should be interpreted as “X,” or “Y,” or “X and Y.” Similarly, “at least one of X or Y” should be interpreted as “X,” or “Y,” or “both X and Y.”

The words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

As used herein, the terms “food,” “food product” and “food composition” mean a product or composition that is intended for oral ingestion by a human or other mammal (e.g., does not contain any plastic) and comprises at least one nutrient for the human or other mammal. “Mammal” includes but is not limited to rodents; aquatic mammals; domestic animals such as dogs and cats; farm animals such as sheep, pigs, cows and horses; and humans. The term “mammal” also applies to any animals that are capable of the effect exhibited or intended to be exhibited by the context of the passage.

As used herein, “infant” means a human child up to the age of one year after birth. “Toddler” means a human child from the age of one to five years of age after birth. “Child” encompasses both infants and toddlers.

As used herein, a “real food” or a “whole food” is understood to mean a food typically ingested by an individual in a normal daily diet when the food is in its natural or prepared state as opposed to any reduced components of the food. For example, a whole food may include any known fruits, vegetables, grain, meats or sources of protein.

“Nutritional compositions” and “nutritional products,” as used herein, include any number of food ingredients and possibly optional additional ingredients based on a functional need in the product and in full compliance with all applicable regulations. The optional ingredients may include, but are not limited to, conventional food additives, for example one or more, acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount.

As used herein, “complete nutrition” means nutritional products that contain sufficient types and levels of macronutrients (protein, fats and carbohydrates) and micronutrients to be sufficient to be a sole source of nutrition for the human child to which it is administered. As used herein, “incomplete nutrition” are nutritional products that do not contain sufficient levels of macronutrients (protein, fats and carbohydrates) or micronutrients to be sufficient to be a sole source of nutrition for the human child to which it is administered.

As used herein, a “snack” food product refers to one or more food products that are smaller in portion size than a meal (i.e., breakfast, lunch and dinner) and/or are intended to be consumed between normal meals of breakfast, lunch and dinner. For example, a snack can have an energy content of 45 kcal per serving or less. A snack typically provides incomplete nutrition, although some embodiments of a snack can optionally instead provide complete nutrition.

The term “edible chew” means a food product, preferably a nutritional composition and preferably a snack, that requires a longer chewing time before the product can be ingested, relative to a standard comestible product. Preferably the edible chew requires at least five minutes of chewing by a human child before the portion of the edible chew intended for consumption is ingested, for example ten minutes to thirty minutes of chewing by a human child before the portion of the edible chew intended for consumption is ingested or even twenty minutes to thirty minutes of chewing by a human child before the portion of the edible chew intended for consumption is ingested.

As used herein, the phrase “dietary recommended intake” is meant to include the nutrition recommendations introduced in 1997 by the Institute of Medicine; used in the U.S. and Canada. As used herein, the phrase “dietary recommended values” is meant to include the nutrition recommendations set in 1991 by the Department of Health of the UK. As used herein, “estimated average requirement” is meant to include the nutrient level expected to meet the needs of 50% of the individuals in an age group. As used herein, “recommended dietary allowance” is meant to include the nutrient level set to meet the needs of 97 to 98% of individuals, or otherwise defined as the average plus 2 standard deviations (goal intake level). As used herein, “reference nutrient intake” is meant to include the nutrient level set to meet the needs of 97 to 98% of individuals, or otherwise defined as the average plus 2 standard deviations (goal intake level).

As used herein, the phrases “tolerable upper limit” and “upper limit” are meant to include the maximum nutrient level that will likely pose no risk of adverse events.

As used herein, “long term administration” refers to continuous administration for at least 6 weeks. “Short term administration” means continuous administrations for less than 6 weeks.

As used herein, the terms “treat” and “treatment” mean to administer a composition as disclosed herein to a subject having a condition in order to lessen, reduce or improve at least one symptom associated with the condition and/or to slow down, reduce or block the progression of the condition. The terms “treatment” and “treat” include both prophylactic or preventive treatment (that prevent and/or slow the development or progression of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

The terms “prevent” and “prevention” mean to administer a composition as disclosed herein to a subject is not showing any symptoms of the condition to reduce or prevent development of at least one symptom associated with the condition. Furthermore, “prevention” includes reduction of risk, incidence and/or severity of a condition or disorder.

As used herein, “effective amount” is an amount that prevents a deficiency, treats a disease or medical condition in an individual or, more generally, reduces symptoms, manages progression of the diseases or provides a nutritional, physiological, or medical benefit to the individual. A treatment can be patient- or doctor-related.

The relative terms “improved,” “increased,” “enhanced” and the like refer to the condition of a human child consuming the edible chew disclosed herein in the context of a diet appropriate for the age and gender of the human child, relative to a human child that consumes the same diet appropriate for their age and gender but without the edible chew disclosed herein.

### Embodiments

The present disclosure provides an edible chew, a method of making the edible chew and a method of using the edible chew. Preferably the edible chew is sized and shaped for a human child such as an infant or a toddler. In an embodiment, the edible chew is substantially homogeneous, has a moisture content of 12-14 wt. %, and a water activity not greater than 0.6.

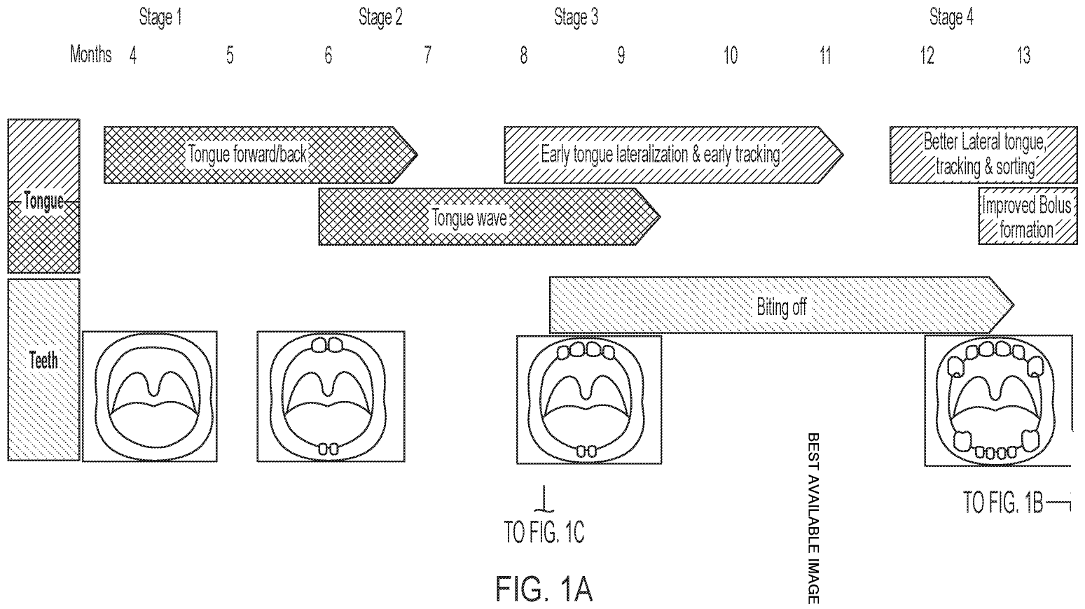

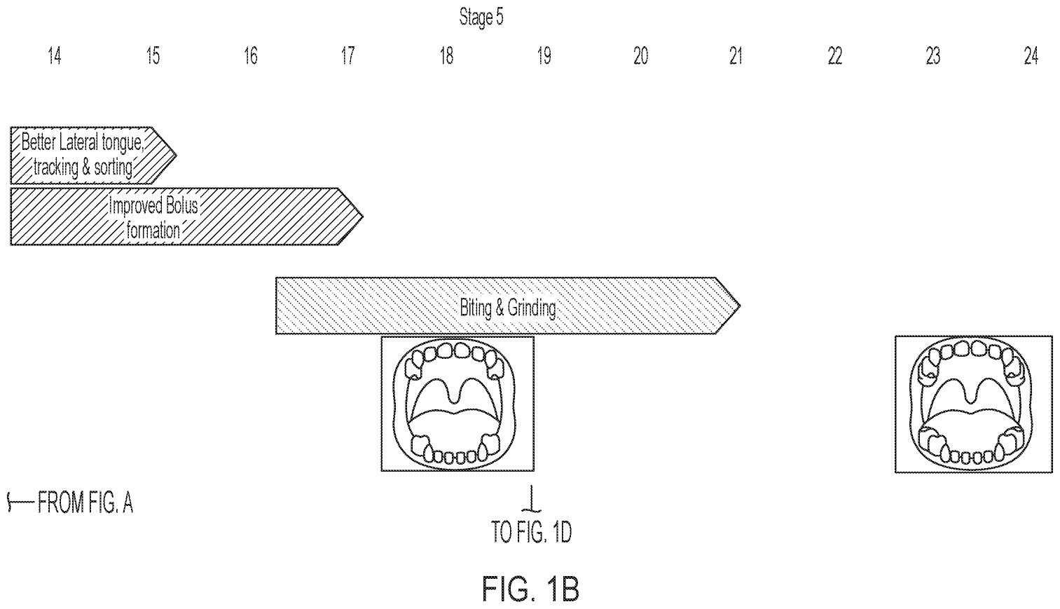

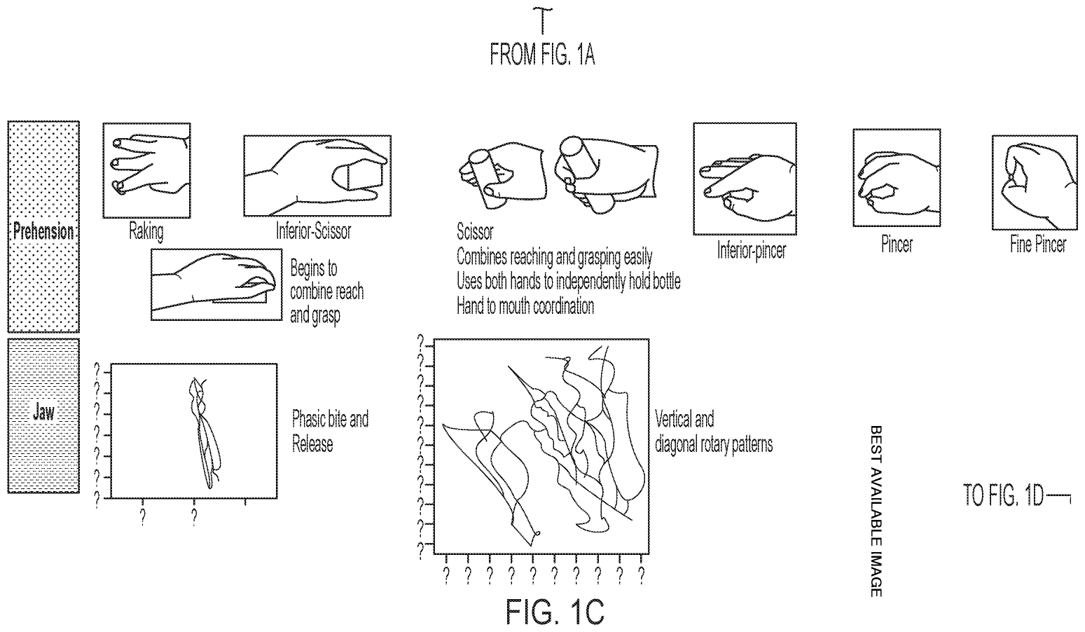

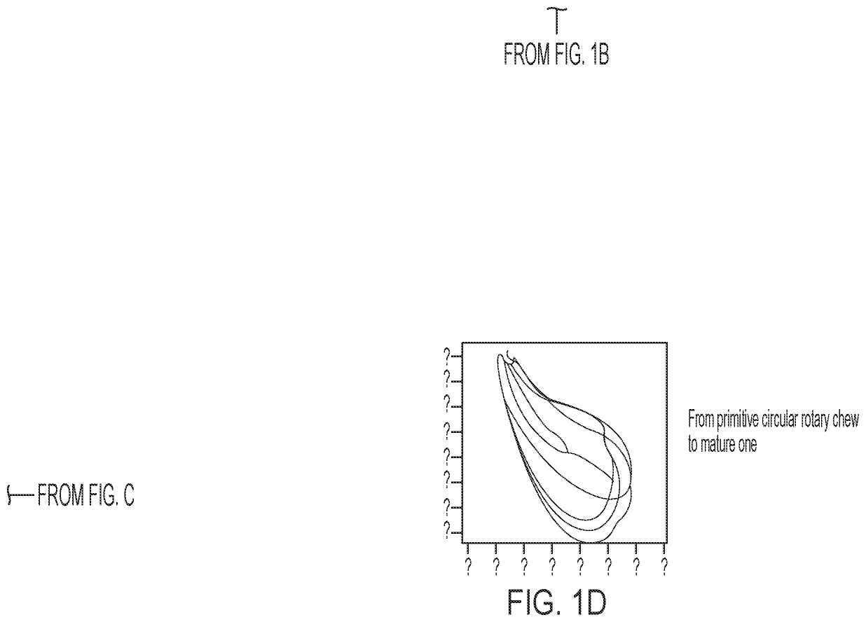

The edible chew can be administered to a human child at any age, including those generally illustrated in FIG. 1. For example, embodiments of the edible chew may be particularly beneficial for a human child from the age of four months after birth to twenty-four months after birth. In this regard, as shown in the figure, the human child will typically be teething during these ages, while also developing their ability to use their tongue and jaw to eat and their ability to use prehension to hold objects and exert hand-to-mouth coordination. Embodiments of the edible chew may support teething (e.g., massage teething gums, pain relief) while assisting the development of tongue usage, jaw control, and prehension skills.

The edible chew is preferably sized and shaped to be held by and consumed by a human child, and the size and the shape of the edible chew can have predetermined dimensions depending on the age of the intended recipient. For example, the edible chew is preferably sized and shaped such that the human child can easily pick up the edible chew with small fingers that are still developing their pincer grasp and other fine motor skills. Once grasped, the edible chew can be readily retained in the child's fingers due to the shape of the edible chew. An example of a size and a shape that fulfills these criteria is a hollow cylinder with an outer diameter of about 15 mm to about 20 mm, for example about 18 mm; an inner diameter; a length of about 50 mm to about 100 mm, for example about 80 mm; and a weight of about 10 g to about 15 g.

Notably, the present disclosure is not limited to administration to a human child having an age depicted in FIG. 1, and a human child who is younger or older is also encompassed by the disclosures herein. Some embodiments of the edible chew can be administered to an older human child no longer teething, for example to soothe and/or calm the child (e.g., relieve one or more of crying, anxiety or other distress) and/or deliver one or more age-appropriate active ingredients discussed in further detail later herein.

In some embodiments, the edible chew is formulated to be chilled or frozen, for example to provide cooling to gums of a teething human child consuming the chilled or frozen edible chew.

The edible chew is preferably administered to the human child as a snack and/or as incomplete nutrition. Nevertheless, some embodiments of the edible chew can provide complete nutrition. The edible chew can be orally administered to the child for short-term administration or long-term administration.

Preferably the edible chew is made by a process comprising extruding pre-gelatinized cereal flour; the pre-gelatinized cereal flour is preferably mixed with a plasticizer in the extruder to form a dough; and the plasticizer is preferably selected from the group consisting of (i) glycerol, (ii) a natural plasticizer that is one or more of glucose syrup, apple juice concentrate, lactose or another short chain carbohydrate, and (iii) a mixture thereof. As discussed in greater detail later herein, a temperature increase at the exit end of the extruder can cause sudden and extensive formation of gas in the dough, resulting in expansion of the dough at both a microscopic and macroscopic level as bubbling gas attempts to escape the material. As a result, the edible chew can be an expanded pre-gelatinized cereal flour matrix.

Non-limiting examples of suitable pre-gelatinized cereal flours include pre-gelatinized rice flour, pre-gelatinized wheat flour, pre-gelatinized corn flour, pre-gelatinized barley flour, pre-gelatinized sorghum flour, and pre-gelatinized millet flour, and combinations thereof. For example, the edible chew can comprise at least about 40 wt. % of the extruded pre-gelatinized cereal flour, preferably at least about 50 wt. % of the extruded pre-gelatinized cereal flour.

The edible chew preferably does not contain any animal by-products, preferably does not contain any yeast, and preferably does not contain any silicon dioxide. In some embodiments, the edible chew does not contain any glycerol and instead contains a natural plasticizer component such as one or more of glucose syrup, apple juice concentrate, lactose or another short chain carbohydrate. The edible chew preferably does not contain any preservatives, for example does not contain any butylated hydroxytoluene (BHT), butylated hydroxyanisole (BHA), sodium benzoate, sodium nitrite, sodium sulfite, sulfur dioxide, propyl paraben, potassium sorbate, calcium propionate or any other preservative.

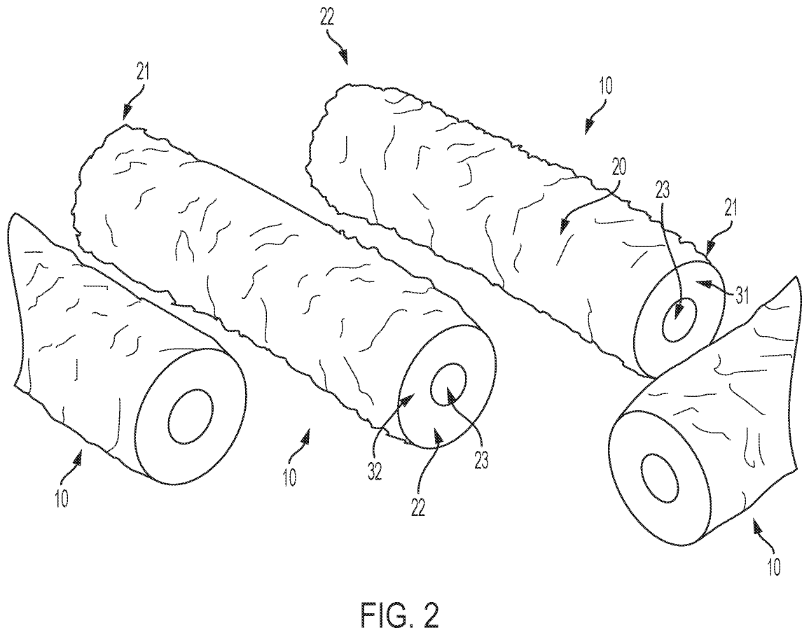

FIG. 2 generally illustrates a plurality of edible chews 10 according to the present disclosure. As shown in the figure, an embodiment of the edible chew 10 is a single unitary piece comprising a body 20 comprising a first end 21 presenting a first substantially planar surface 31 (“first end surface 31”) and further comprises a second end 22 presenting a second substantially planar surface 32 (“second end surface 32”). The first and second end surfaces 31,32 can be substantially the same shape as each other and substantially the same size as each other. The first and second end surfaces 31,32 can be essentially parallel to each other and define the length of the edible chew 10 (e.g., the edible chew 10 can terminate at the first end surface 31 and at the second end surface 32). The body 20 of the edible chew 10 preferably has a substantially cylindrical outer surface that can extend from the first end surface 31 to the second end surface 32 and that can have an essentially constant outer diameter along the entire length of the body 20.

In a particular non-limiting embodiment, the edible chew 10 can consist essentially of or consist of the substantially cylindrical body 20 such that the edible chew 10 does not have any outer surface that significantly extends outward or inward from the substantially constant outer diameter, the first substantially planar surface, or the second substantially planar surface (e.g., no outer surface extending more than about 2 mm, preferably no more than about 1 mm, from the substantially constant outer diameter, the first substantially planar surface, or the second substantially planar surface). For example, the edible chew 10 preferably does not have any large ridges, large grooves, or large undulations. This embodiment of the edible chew 10 can help ensure safety for a human child consuming the edible chew 10.

Preferably the body 20 has a hollow center 23, for example a substantially cylindrical cavity, that can optionally extend through the entire length of the body 20 such that the body 20 has an essentially constant inner diameter along the entire length of the body 20. The first end surface 31 can comprise a first opening, the second end surface 32 can comprise a second opening, and the hollow center 23 (e.g., substantially cylindrical cavity) can be exposed to the exterior of the edible chew 10 through the first and second openings.

In some embodiments, optionally one or more fillings can be positioned in at least a portion of the hollow center 23 (e.g., substantially cylindrical cavity), for example in the substantial entirety of the cavity. In a particular non-limiting embodiment, the edible chew 10 can consist essentially of or consist of the substantially cylindrical body 20 and the one or more fillings in the hollow center 23 such that the edible chew 10 does not have any outer surface that significantly extends outward or inward from the substantially constant outer diameter, the first substantially planar surface, or the second substantially planar surface (e.g., no outer surface extending more than about 2 mm, preferably no more than about 1 mm, from the substantially constant outer diameter, the first substantially planar surface, or the second substantially planar surface). For example, the edible chew 10 preferably does not have any large ridges, large grooves, or large undulations. This embodiment of the edible chew 10 can also help ensure safety for a human child consuming the edible chew 10.

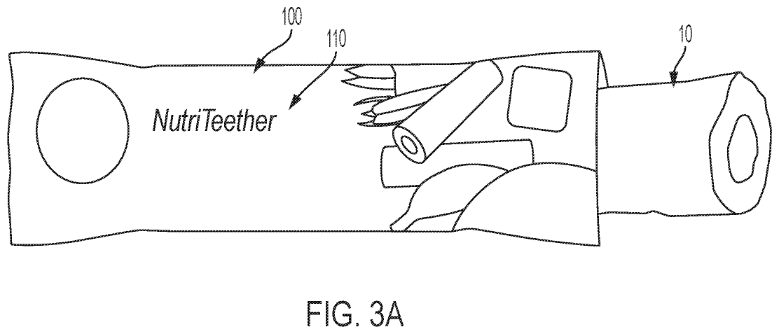

As shown in FIGS. 3A and 3B, the edible chew 10 can be individually packaged, for example in packaging 100 that can be sealed to retain the texture of shelf life of the edible chew 10. The packaging 100 can include storage instructions and/or safe use instructions, such as age and milestone depictions. For example, the packaging 100 can include indicia 110, such as text and/or graphics, that indicates one or more of: the edible chew 10 should be administered to a human child who is seated and supervised, supervised use of the edible chew 10 is required, and the edible chew 10 should be disposed when the edible chew 10 becomes the same size as the child's hand. Optionally a packaging insert in the interior of the packaging 100 can provide teething information and/or product information. The usage instructions on the packaging 100 can state that the packaging 100 should be opened shortly before to the time of consumption of the edible chew 10.

A plurality of individually packaged edible chews can be provided to the consumer in a container, for example a box that holds a plurality of individually packaged edible chews, such as twenty-four packaged edible chews. Preferably the plurality of edible chews are substantially identical to each other, for example substantially the same size, the same shape and the same texture relative to each other.

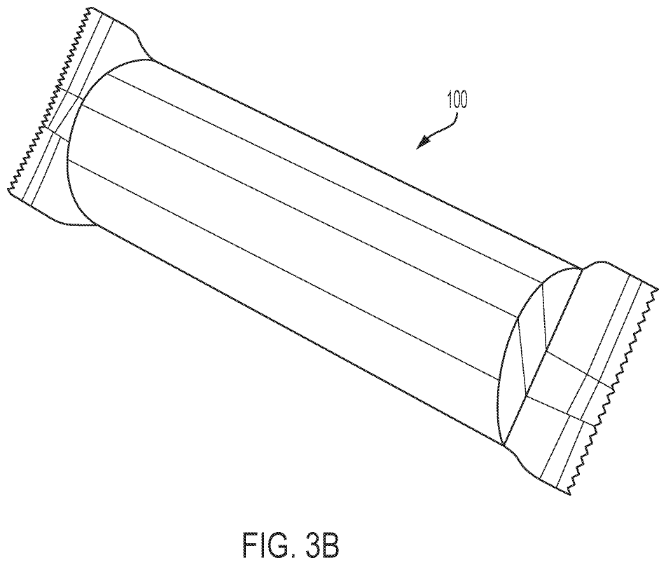

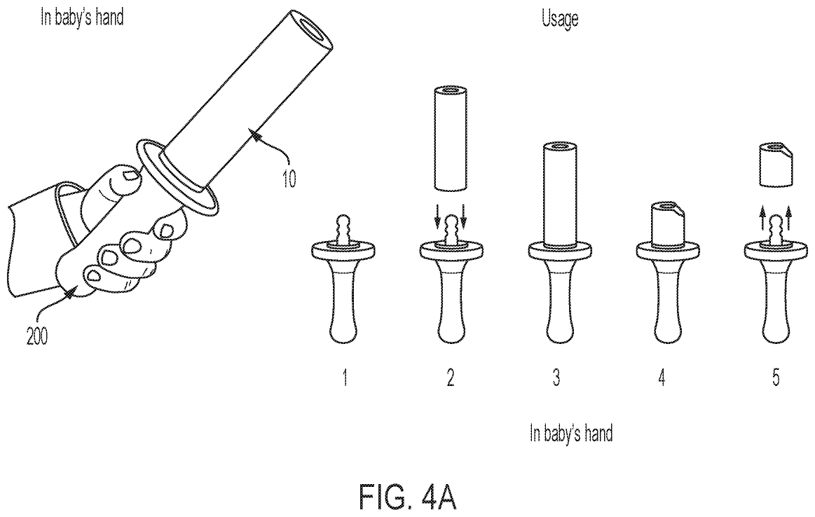

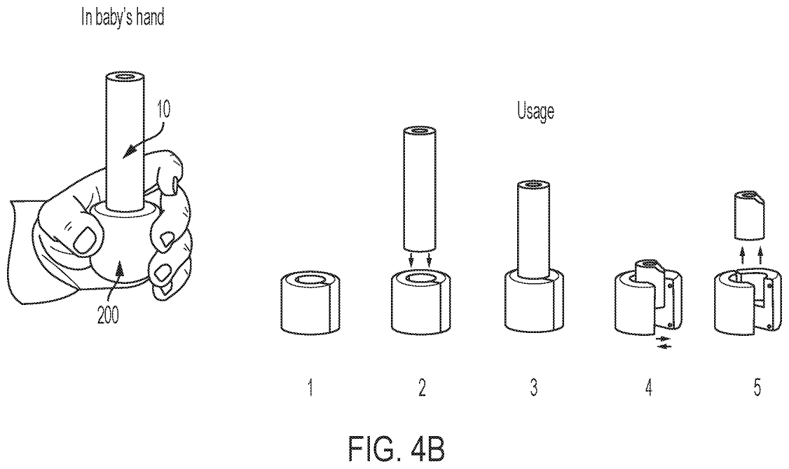

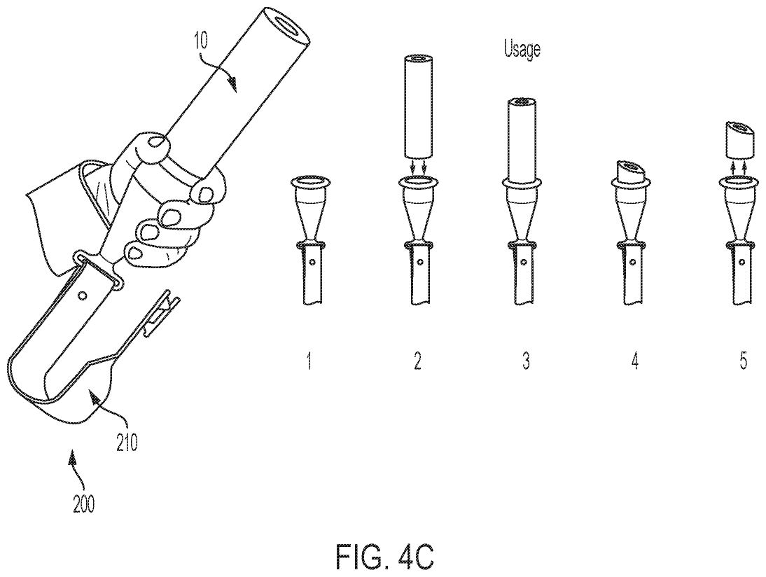

As shown in FIGS. 4A, 4B and 4C, the edible chew 10 can be used with a holder 200. The holder 200 can be configured to attach to the edible chew 10, preferably to prevent a human child eating the edible chew 10 from eating the last piece of the edible chew 10. The holder 200 is preferably sized and shaped to be held by a human child while the edible chew 10 is positioned in the holder 200 and/or on the holder 200.

The holder 200 preferably attaches to the edible chew 10 such that a human adult can remove the edible chew 10 from the holder 200 using only their hands, e.g., without using tools, but attaches to the edible chew 10 fixedly enough that a human child cannot remove the edible chew 10 from the holder 200 using only their hands.

As shown in the figures, a method of using the holder 200 can comprise one or more of the following steps: Step 1—human adult has holder 200, Step 2—the human adult positions the edible chew 10 in the holder 200 and/or on the holder 200, Step 3—the human child eats a top portion of the edible chew 10 while the holder 200 retains the bottom portion of the edible chew 10, Step 4—the human adult takes the holder 200 from the human child while the bottom portion of the edible chew 10 is attached to the holder 200, and Step 5—the human adult removes the bottom portion of the edible chew 10 from the holder 200 and disposes the bottom portion of the edible chew 10 (preferably without disposing the holder 200, particularly in an embodiment in which the holder 200 is reusable).

In the embodiment shown in FIG. 4A, the holder 200 can comprise a handle for the human child's hand and can further comprise a stick configured to insert at least partially into the hollow center 23 of the edible chew 10. Attaching the edible chew 10 to the holder 200 can comprise inserting the stick of the holder 200 into the hollow center 23 of the bottom portion of the edible chew 10 (Step 2), and removing the bottom portion of the edible chew 10 from the holder 200 can comprise removing the stick of the holder 200 from the hollow center 23 of the bottom portion of the edible chew 10 (Step 5). Preferably the stick has a smaller outer diameter than the handle, and optionally the holder 200 further comprises a guard between the stick and the handle that has an outer diameter larger than that of the stick and that of the handle.

In the embodiment shown in FIG. 4B, the holder 200 can comprise a socket configured to receive the bottom portion of the edible chew 10. Attaching the edible chew 10 to the holder 200 can comprise inserting the bottom portion of the edible chew 10 into the socket (Step 2), and removing the bottom portion of the edible chew 10 from the holder 200 can comprise removing the bottom portion of the edible chew 10 from the socket (Step 5).

Preferably this embodiment of the holder 200 comprises at least two parts that delimit the socket and are movable relative to each other such that a closed configuration holds the bottom portion of the edible chew 10 in the socket and an open configuration releases the bottom portion of the edible chew 10 from the socket, for example by one or more of the at least two parts pivoting relative to another part.

In the embodiment shown in FIG. 4C, at least a portion of the holder 200 can form a tube into which the edible chew 10 can at least partially insert. Attaching the edible chew 10 to the holder 200 can comprise inserting the bottom portion of the edible chew 10 into the tube (Step 2), and removing the bottom portion of the edible chew 10 from the holder 200 can comprise removing the bottom portion of the edible chew 10 from the tube (Step 5).

Preferably the tube has an inner diameter slightly larger than the outer diameter of the edible chew 10 (e.g., about 0.1% larger than the outer diameter of the edible chew 10, about 0.5% larger than the outer diameter of the edible chew 10 or about 1.0% larger than the outer diameter of the edible chew 10) such that the bottom portion of the edible chew 10 snugly fits within the holder 200 and is securely held therein to prevent removal by the human child.

The holder 200 is not limited to these particular exemplary embodiments, and the holder 200 can be any device that attaches to the edible chew 10 and prevents the human child from eating the bottom portion of the edible chew 10. In some embodiments of the holder 200, an attachment member 210 is configured to connect to the holder 200 and to connect to clothing of the human child, for example a bib worn by the human child, to ensure the edible chew 10 maintains proximity to the human child. A non-limiting example of a suitable attachment member 210 is a strap that connects to the holder 200 at one end of the strap and comprises a clip at the other end of the strap to connect to clothing of the human child, for example a bib worn by the human child. FIG. 4C generally illustrates the attachment member 210, but the attachment member 210 can be configured to be used with any embodiment of the holder 200.

In some embodiments, the holder 200 can be provided with the edible chew 10 (e.g., a plurality of the edible chews) in a kit. A “kit” means that the components of the kit are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof.

Referring again to the edible chew generally, some embodiments of the edible chew can further comprise an active ingredient in addition to the extruded pre-gelatinized cereal flour, for example one or more of chamomile, lavender, a sugar alcohol/polyol (preferably not glycerol), a cooling sensate such as menthol, a prebiotic, a probiotic, a protein, an amino acid, an essential oil, a homeopathic ingredient, a pharmaceutical compound, or a micronutrient such as a vitamin and/or a mineral.

The term “vitamin” includes any fat-soluble or water-soluble organic substance essential for normal growth and activity of the human body and obtained naturally from plant and animal foods. Additionally or alternatively, the at least a portion of the vitamins can be synthetically made, pro-vitamins, derivatives or analogs.

Non-limiting examples of suitable vitamins for the edible chew include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), Vitamin B12 (various cobalamins, commonly cyanocobalamin in vitamin supplements), Vitamin C, Vitamin D, Vitamin E, Vitamin K, folic acid, biotin or mixtures thereof.

The term “minerals” includes any abiogenic substance essential for normal growth and activity of the human body. Non-limiting examples of suitable vitamins for the edible chew include boron, calcium, chromium, copper, iodine, iron, magnesium, manganese, molybdenum, nickel, phosphorus, potassium, selenium, silicon, tin, vanadium, zinc, or mixtures thereof.

A preferred embodiment of the edible chew further comprises at least one of Vitamin B1, Vitamin D or calcium in addition to the extruded pre-gelatinized cereal flour. For example, the edible chew can comprise about 70 mg of calcium and/or about 20% of the dietary recommended intake of calcium for an infant or toddler, the dietary recommended value of calcium for an infant or toddler, the estimated average requirement of calcium for an infant or toddler, the recommended dietary allowance of calcium for an infant or toddler or the reference nutrient intake of calcium for an infant or toddler.

In an embodiment, the edible chew can include at least two sources or forms of a particular vitamin or mineral to represent a mixture of vitamin and mineral sources as found in a mixed diet. A mixture of at least two sources or forms of a particular vitamin or mineral may also be protective for an individual who has difficulty absorbing a specific form. In this regard, a mixture of at least two sources or forms of a particular vitamin or mineral may increase uptake through use of different transporters (e.g., zinc, selenium) or may offer a specific health benefit. As a non-limiting example, Vitamin E has several forms, the most commonly consumed and researched being tocopherols (alpha, beta, gamma, delta) and, less commonly, tocotrienols (alpha, beta, gamma, delta), which all vary in biological activity. The structure of tocotrienols allows them to more freely move around the cell membrane; several studies report various health benefits related to cholesterol levels, immune health, and reduced risk of cancer development. A mixture of tocopherols and tocotrienols can cover the range of biological activity.

The term “microorganism” is meant to include the bacterium, yeast and/or fungi itself; a cell growth medium with the microorganism; and/or a cell growth medium in which microorganism was cultivated. As used herein, a “food grade micro-organism” is a micro-organism generally regarded as safe for use in food.

As used herein, “probiotics” are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See Salminen S, Ouwehand A. Benno Y. et al., “Probiotics: how should they be defined?” Trends Food Sci. Technol., 10(3):107-10 (1999). In general, these micro-organisms are believed to inhibit or influence the growth and/or metabolism of pathogenic bacteria in the intestinal tract. The probiotics may also activate the immune function of the host.

Non-limiting examples of suitable probiotics for the edible chew include Aerococcus, Aspergillus, Bacillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or mixtures thereof.

As used herein, a “non-replicating” microorganism means that no viable cells and/or colony forming units can be detected by classical plating methods. Such classical plating methods are summarized in the microbiology book: James Monroe Jay, et al., “Modern food microbiology,” 7th edition, Springer Science, New York, N. Y. p. 790 (2005). Typically, the absence of viable cells can be shown as follows: no visible colony on agar plates or no increasing turbidity in liquid growth medium after inoculation with different concentrations of bacterial preparations (“non-replicating” samples) and incubation under appropriate conditions (aerobic and/or anaerobic atmosphere for at least 24 hours). For example, Bifidobacteria such as Bifidobacterium longum, Bifidobacterium lactis and Bifidobacterium breve, or Lactobacilli, such as Lactobacillus paracasei or Lactobacillus rhamnosus, may be rendered non-replicating by heat treatment, in particular low temperature/long time heat treatment.

As used herein, a “prebiotic” is a food substance that selectively promotes the growth of beneficial bacteria or inhibits the growth or mucosal adhesion of pathogenic bacteria in the intestines. Prebiotics are not inactivated in the stomach and/or upper intestine or absorbed in the gastrointestinal tract of the person ingesting them, but they are fermented by the gastrointestinal microflora and/or by probiotics. Prebiotics are, for example, defined by Glenn R. Gibson and Marcel B. Roberfroid, “Dietary Modulation of the Human Colonic Microbiota: Introducing the Concept of Prebiotics,” J. Nutr. 1995 125: 1401-1412.

Non-limiting examples of suitable prebiotics for the edible chew include acacia gum, alpha-glucan, arabinogalactans, beta-glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or mixtures thereof.

As used herein, a “synbiotic” is a supplement that contains both a prebiotic and a probiotic that work together to improve the microflora of the intestine.

The term “protein” refers to any composition that includes one or more single amino acids (monomers), two or more amino acids joined together by a peptide bond (dipeptide, tripeptide, or polypeptide), collagen, a precursor, a homolog, an analog, a mimetic, a salt, a prodrug, a metabolite, a fragment thereof or mixtures thereof. Non-limiting examples of suitable amino acids for the edible chew include alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or mixtures thereof.

Proteins can contain amino acids other than the twenty naturally occurring amino acids; and many amino acids, including the terminal amino acids, may be modified in a given polypeptide, either by natural processes such as glycosylation and other post-translational modifications or by chemical modification techniques which are well known in the art. Among the known modifications which may be present in polypeptides suitable for inclusion in the edible chew include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

Non-limiting examples of suitable proteins for the edible chew include dairy-based proteins, plant-based proteins, animal-based proteins, artificial proteins and mixtures thereof. Dairy-based proteins include, for example, casein, caseinates (e.g., all forms including sodium, calcium and potassium caseinates), casein hydrolysates, whey (e.g., all forms including concentrate, isolate and demineralized), whey hydrolysates, milk protein concentrate, and milk protein isolate. Plant-based proteins include, for example, soy protein (e.g., all forms including concentrate and isolate); pea protein (e.g., all forms including concentrate and isolate), canola protein (e.g., all forms including concentrate and isolate); and other plant proteins such as wheat and fractionated wheat proteins, corn and its fractions including zein, rice, oat, potato, peanut, green pea powder, green bean powder, and any proteins derived from beans, lentils, and pulses. Animal-based proteins are preferably selected from the group consisting of beef, poultry, fish, lamb, seafood, or mixtures thereof.

In addition to the pre-gelatinized cereal flour, the edible chew may also include other beneficial or functional ingredients, for example, a carbohydrate, a fat, a fiber or a mixture thereof.

Non-limiting examples of suitable carbohydrates for the edible chew include one or more of: (i) monosaccharides such as trioses, for example ketotriose (dihydroxyacetone) and aldotriose (glyceraldehyde); tetroses, for example ketotetroses (e.g., erythrulose) and aldotetroses (e.g., erythrose and threose); pentoses, for example ketopentoses (e.g., ribulose and xylulose) and aldopentoses (e.g., ribose, arabinose, xylose and lyxose); deoxy sugars, for example deoxyribose, fucose, fuculose and rhamnose; hexoses, for example ketohexoses (e.g., psicose, fructose, sorbose, tagatose); aldohexoses (e.g., allose, altrose, glucose, mannose, gulose, idose, galactose and talose), heptoses (e.g., sedoheptulose); octoses; and nonoses, for example neuraminic acid; (ii) disaccharides such as sucrose, lactose, maltose, trehalose, turanose, cellobiose, kojiboise, sakebiose, isomaltose, and palatinose; (iii) trisaccharides such as melezitose and maltotriose; (iv) oligosaccharides such as corn syrups and maltodextrin; and (v) polysaccharides such as glucans, for example dextrin, dextran and beta-glucan; glycogen; mannan; galactan; and starch, for example those from corn, wheat, tapioca, rice, and potato, including amylose and amylopectin.

Any starch can be natural or modified or gelatinized or a mixture thereof. Non-limiting examples of suitable carbohydrates for the edible chew also include a source of sweeteners such as honey, maple syrup, glucose (dextrose), corn syrup, corn syrup solids, high fructose corn syrups, crystalline fructose, juice concentrates, and crystalline juice. In a preferred embodiment, any carbohydrate in the edible chew (in addition to the pre-gelatinized cereal flour) is selected from the group consisting of sucrose, lactose, glucose, fructose, corn syrup solids, maltodextrin, modified starch, amylose starch, tapioca starch, corn starch, and mixtures thereof.

The edible chew can optionally comprise fiber, a source of fiber, or a blend of different types of fiber, such as a mixture of soluble and insoluble fibers. Soluble fibers may include, for example, one or more of fructooligosaccharides, acacia gum, and inulin. Insoluble fibers may include, for example, pea outer fiber.

Non-limiting examples of a source of fat for the edible chew include any suitable fat or fat mixture. For example, the fat may include, but is not limited to, vegetable fat (such as olive oil, corn oil, sunflower oil, rapeseed oil, hazelnut oil, soy oil, palm oil, coconut oil, canola oil, or lecithins) and animal fat (such as milk fat). The source of fat can optionally provide one or more ω-3 fatty acids such as a-linolenic acid (“ALA”), docosahexaenoic acid (“DHA”) and eicosapentaenoic acid (“EPA”). Non-limiting examples of sources of ω-3 fatty acids suitable for the edible chew include fish oil, krill, poultry, eggs, or other plant or nut sources such as flax seed, walnuts, almonds, algae, and modified plants.

As another example, the edible chew may include a “nucleotide,” which is a subunit of deoxyribonucleic acid (“DNA”) or ribonucleic acid (“RNA”). A nucleotide is an organic compound having a nitrogenous base, a phosphate molecule, and a sugar molecule (deoxyribose in DNA, ribose in RNA). Individual nucleotide monomers (single units) are linked together to form polymers or long chains. Exogenous nucleotides are specifically provided by dietary supplementation. The exogenous nucleotide can be in a monomeric form such as, for example, 5′-Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”), or combinations thereof. The exogenous nucleotide can also be in a polymeric form such as, for example, an intact RNA. The edible chew can include multiple sources of the polymeric form of the exogenous nucleotide such as, for example, yeast RNA.

As yet another example, the edible chew may include an antioxidant such as Vitamin A, beta-carotene (a Vitamin A precursor), Vitamin B₁, Vitamin B₆, Vitamin B₁₂, Vitamin C, Vitamin D, Vitamin E, zeaxanthin, carotenoids, coenzyme Q10 (“CoQ10”), flavonoids, glutathione, Goji (wolfberry), hesperidin, lactowolfberry, lignan, lutein, lycopene, polyphenols, selenium or mixtures thereof.

As still another example, the edible chew may include a “phytochemical” or “a phytonutrient,” which are non-nutritive compounds that are found in many foods. Phytochemicals are functional foods that have health benefits beyond basic nutrition, and are health promoting compounds that come from plant sources. “Phytochemicals” and “Phytonutrients” refers to any chemical produced by a plant that imparts one or more health benefit on the user. Non-limiting examples of phytochemicals and phytonutrients suitable for the edible chew include:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3 -gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and ω-3, -6, and -9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors;

vi) other organic acids which include oxalic acid, phytic acid (inositol hexaphosphate); tartaric acid; and anacardic acid; or

vii) mixtures thereof.

Other optional ingredients can be added to make the edible chew. For example, the edible chew can optionally include conventional food additives, such as any of, acidulants, additional thickeners, buffers or agents for pH adjustment, chelating agents, colorants, emulsifiers, excipients, flavor agents, minerals, osmotic agents, pharmaceutically acceptable carriers, preservatives, stabilizers, sugars, sweeteners, texturizers, or combinations thereof. The optional ingredients can be added in any suitable amount.

In yet another aspect of the present disclosure, a method of making the edible chew is provided. The method can use pre-gelatinized cereal flour as at least a portion of the starting material, for example by forming a dry mix comprising the pre-gelatinized cereal flour. Non-limiting examples of suitable pre-gelatinized cereal flours include pre-gelatinized rice flour, pre-gelatinized wheat flour, pre-gelatinized corn flour, pre-gelatinized barley flour, pre-gelatinized sorghum flour, and pre-gelatinized millet flour, and combinations thereof. Optionally the dry mix can include other components in addition to the pre-gelatinized cereal flour, for example any food ingredient suitable for consumption by a human child, such as any of the optional additional ingredients disclosed herein. Preferably the method of making the edible chew does not involve using a supercritical fluid.

The pre-gelatinized cereal flour can be dynamically cooked with liquid ingredients in an extruder to form a dough. The liquid ingredients preferably comprise a plasticizer selected from the group consisting of (i) glycerol, (ii) a natural plasticizer, for example one or more of glucose syrup, apple juice concentrate, lactose or another short chain carbohydrate, and (iii) a mixture thereof. The liquid ingredients can be injected into the extruder while the dry mix is introduced therein and/or processed therein. Optionally the liquid ingredients can further comprise ingredients additional to the plasticizer, for example vegetable oil and/or an emulsifier such as monoglycerides.

In the extruder, the pre-gelatinized cereal flour and the other ingredients can mix with each other while shear is imparted within the extruder barrel. Preferably the extruder has a plurality of successive barrels, and the temperature increases in one or more of the barrels adjacent the die through which the cooked dough exits, relative to the prior barrels. For example, the barrel at the exit end of the extruder can be heated to a temperature of about 120° C., and one or more of the prior barrels can be heated to a temperature of about 80° C. The temperature increase at the exit end of the extruder can cause sudden and extensive formation of gas in the dough, resulting in expansion of the dough at both a microscopic and macroscopic level as bubbling gas attempts to escape the material.

As a non-limiting example, the initial barrels (e.g., the 6 first barrels) are not heated, for example by having the temperature therein set to about 25° C., although the temperature of these barrels is not necessarily the set temperature because the cooling capacity is not always sufficient. The temperature of the last barrels (e.g., the last four barrels) may be 80-120° C. for low product throughput (100-150 kg/h) and may be lower for higher throughput (high shearing due to screw speed). Moreover, the temperature of the last barrels can be lower if the last part of the screw profile therein is stronger. The average pressure at the front plate can be about 75 bars at about 115 kg/h on a one rope die; higher pressure (e.g. about 95 bars) can be reached at higher throughput 400 kg/h on a four rope die. The average SME value can be 400-450 KJ/kg on dry basis.

Preferably the extrusion die has a profile configured to form the hollow center of the edible chew. For example, the extrusion die can comprise an opening and can comprise a mandrel positioned at least partially positioned in the opening, and the dynamically cooked dough comprising gelatinized cereal flour and plasticizer can be extruded through the gap between the inner diameter of the opening of the extrusion die and the outer diameter of the mandrel, thereby forming extrudate with a hollow center.

Upon discharge from the extrusion die, the extrudate can undergo further processing to achieve the embodiments of the edible chew disclosed herein, for example cooling the extrudate (e.g., in a cooling device or at ambient temperature) and/or cutting the extrudate into pieces of a predetermined size (e.g., with a cutting device optionally mounted to the extruder).

Accordingly, an embodiment of a method of making the edible chew comprises preparing a dry mix comprising a pre-gelatinized cereal flour; metering the dry mix into an extruder comprising an extrusion die; injecting liquid ingredients comprising a plasticizer into the extruder to form a dough from the pre-gelatinized cereal flour, the plasticizer preferably selected from the group consisting of (i) glycerol, (ii) a natural plasticizer such as one or more of glucose syrup, apple juice concentrate, lactose or another short chain carbohydrate, and (iii) mixtures thereof; promoting nucleation of the dough by subjecting the dough to a combination of shear and a temperature in a section of the extruder adjacent to the extrusion die to form a heated and sheared dough, the temperature in the section adjacent to the extrusion die is higher than a temperature of at least one previous section of the extruder; and directing the heated and sheared dough through an opening of the extrusion die. In an embodiment, the temperature in the section of the extruder adjacent to the extrusion die is at least 120° C.

In an embodiment, a bicarbonate can be used, for example sodium bicarbonate, preferably by inclusion ion the dry mix with the pre-gelatinized cereal flour. As non-limiting example, sodium bicarbonate can be 0.2 to 1.5 wt. % of the dry mix, preferably 0.5 to 1.5 wt. % of the dry mix. Without being bound by theory, the inventors believe that the carbon dioxide released from the bicarbonate submitted to heat and/or acid promote nucleation (more nucleation sites) and helps to decrease the size of the foam cells that form in the material during extrusion.

Some embodiments of the method achieve a low density of the edible chew (e.g., a density of about 1.0 Kg/L or less, such as about 1.0 Kg/L, or even about 0.9 Kg/L or less, such as about 0.9 Kg/L) by incorporating air into the dough during extrusion to aerate the dough. In such embodiments, the expansion can be primarily in a radial direction, although the use of a bicarbonate such as sodium bicarbonate can result in combined axial and radial expansion in these embodiments.

In these low density embodiments, the extruder can comprise a barrel comprising a vent that is open to the atmosphere and comprising a vent-stuffer, and the dough in the venting barrel must be viscous enough to entrap air. A reverse element can be positioned after the venting barrel to maintain the dough for an extended period of time in the venting barrel. The reverse element can be a reverse screw element or any element that generates backpressure in the extruder. The extended period of time spent by the dough in the venting barrel in the presence of the mixing elements therein can ensure an efficient mixing and/or whipping of the dough. The vent in the venting barrel that is open to the atmosphere can allow air to penetrate into the extruder and push the dough, potentially coming through the vent, back into the extruder. However, in a preferred embodiment, forced gas injection is not used.

This “whipping” process promotes nucleation such that a very foamy and light dough is obtained. The phenomenon is visible right after the front plate, on the opposite of the previous alternatives where the bubbles mainly grow at the die exhaust. Product surface is smooth, and product shape is well-defined, because of the very tiny bubble size. Addition of a bicarbonate such as sodium bicarbonate to the dry mix can increase the nucleation even further.

Accordingly, another embodiment of a method of making the edible chew comprises preparing a dry mix comprising a pre-gelatinized cereal flour; metering the dry mix into an extruder comprising an extrusion die and a barrel comprising a vent and a vent-stuffer; adding a plasticizer into the extruder to form a dough from the pre-gelatinized cereal flour, the plasticizer preferably selected from the group consisting of (i) glycerol, (ii) a natural plasticizer such as one or more of glucose syrup, apple juice concentrate, lactose or another short chain carbohydrate, and (iii) mixtures thereof; using a reverse element of the extruder to extend a time spent by the dough in the barrel comprising the vent and the vent-stuffer; using mixing elements in the barrel comprising the vent and the vent-stuffer to whip the dough and promote nucleation; and promoting further nucleation of the dough by subjecting the dough to a combination of shear and a temperature in a section of the extruder adjacent to the extrusion die to form a heated and sheared dough, the temperature in the section of the extruder adjacent to the extrusion die is higher than a temperature of at least one previous section of the extruder. Extending the time spent by the dough in the first section can provide for a better whipping of the dough.

In an embodiment, this method comprises adding a bicarbonate to the dry mix. The method can optionally comprise aerating the dough in the barrel comprising the vent and the vent-stuffer. In an embodiment, the vent and the vent-stuffer are configured to provide ambient pressure.

Preferably the extrusion die has a profile configured to form the hollow center of the edible chew. For example, the extrusion die can comprise an opening and can comprise a mandrel positioned at least partially positioned in the opening, and the dynamically cooked dough comprising gelatinized cereal flour and plasticizer can be extruded through the gap between the inner diameter of the opening of the extrusion die and the outer diameter of the mandrel, thereby forming extrudate with a hollow center.

Upon discharge from the extrusion die, the extrudate can undergo further processing to achieve the embodiments of the edible chew disclosed herein, for example cooling the extrudate (e.g., in a cooling device or at ambient temperature) and/or cutting the extrudate into pieces of a predetermined size (e.g., with a cutting device optionally mounted to the extruder).

### EXAMPLES

By way of example and not limitation, the following examples are illustrative of various embodiments of the present disclosure. The formulations and processes below are provided for exemplification only, and they can be modified by the skilled artisan to the necessary extent, depending on the special features that are desired.

### Example 1

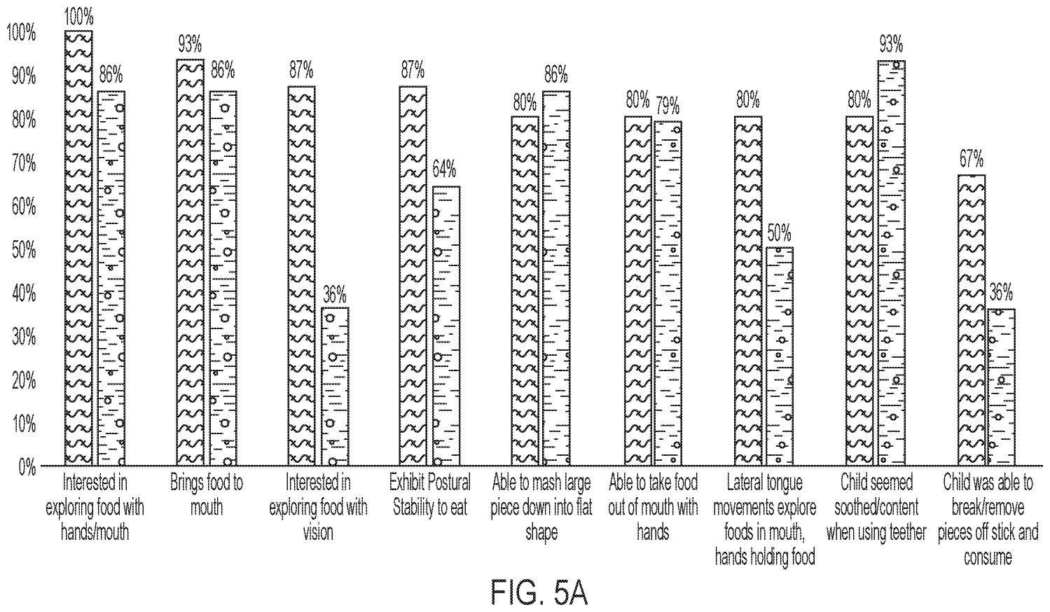

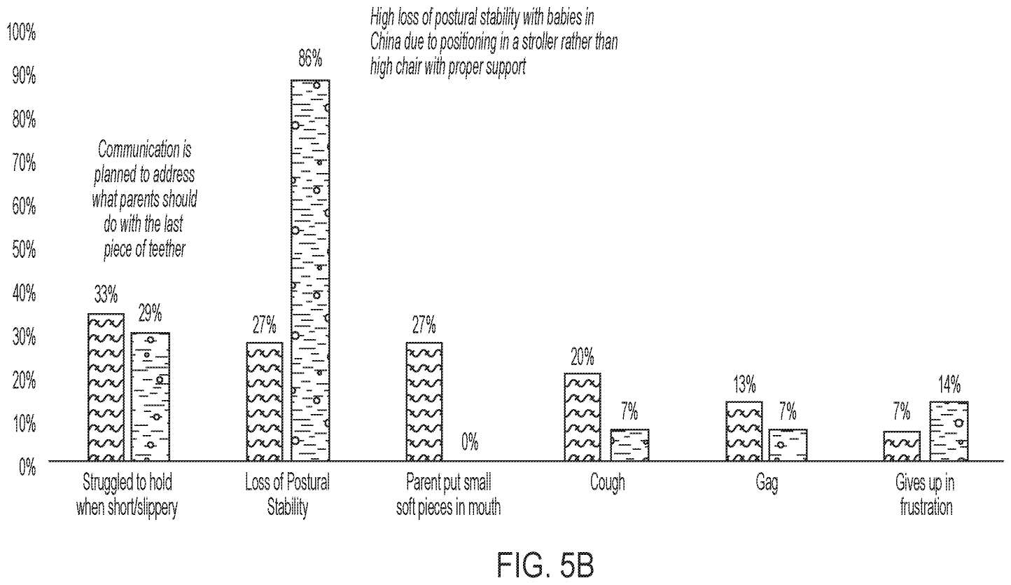

Video observation was conducted with human children aged six months to fifteen months who were given the edible chew according to the present disclosure. FIG. 5A shows that a majority of the children exhibited the feeding skills needed to successfully eat the edible chew. FIG. 5B showing that challenging behaviors were observed in a limited number of the children months, with no choking behaviors observed.

### Example 2

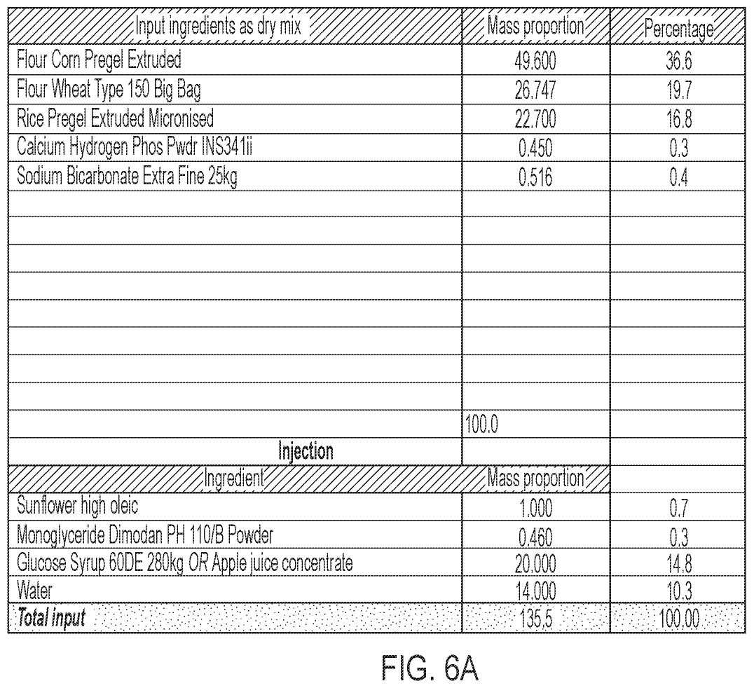

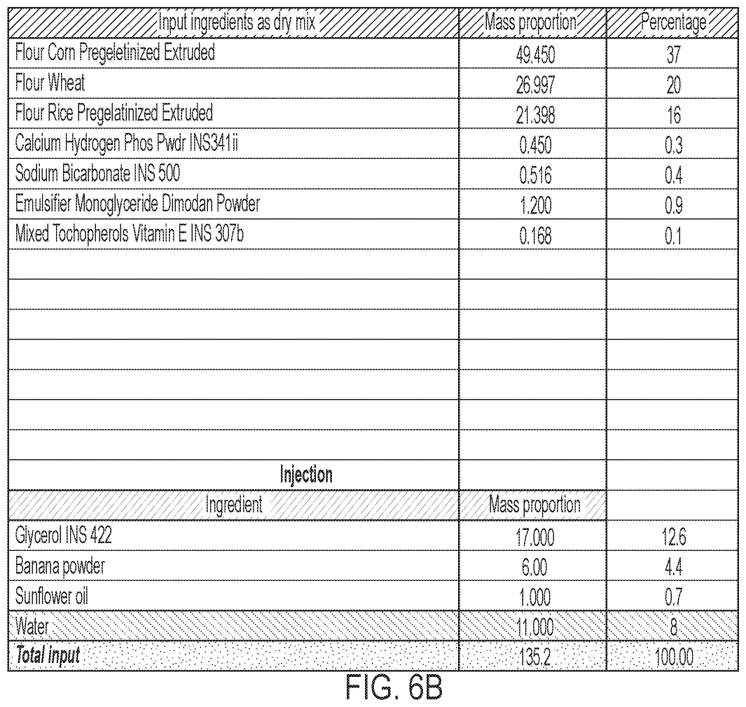

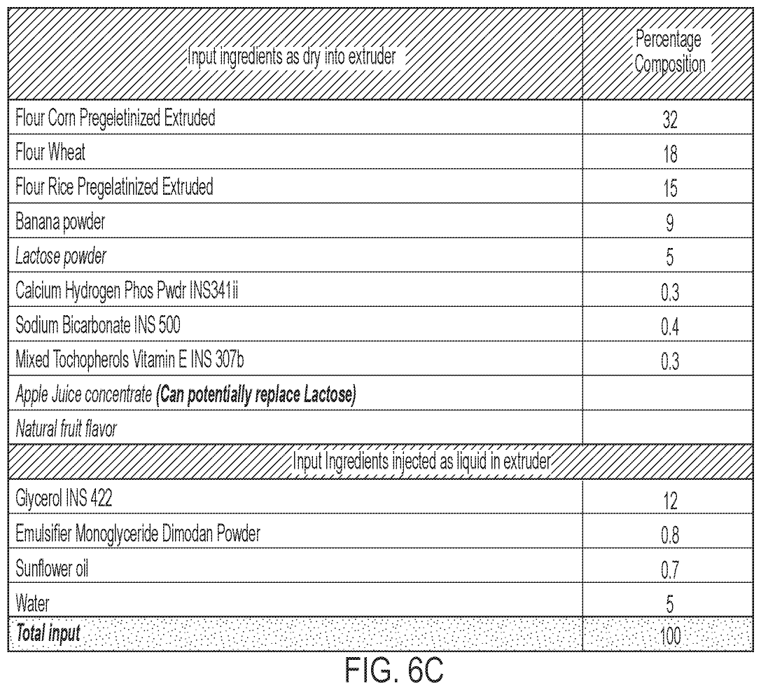

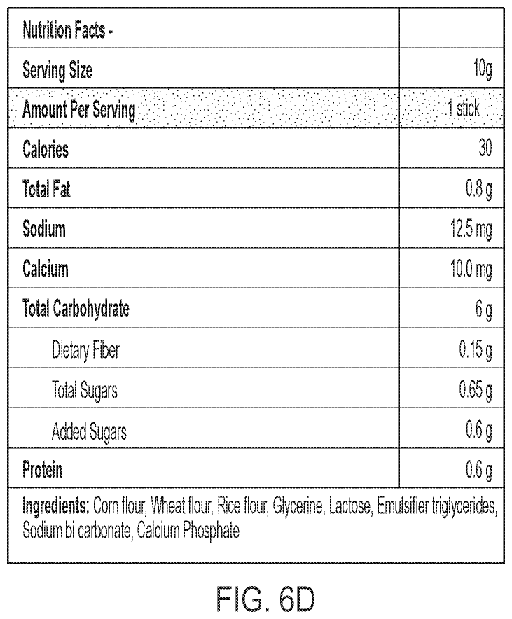

FIG. 6A shows a non-limiting example of a formulation for an embodiment of the edible chew that does not contain glycerol. FIGS. 6B and 6C show non-limiting examples of formulations for an embodiment of the edible chew that contain glycerol, and FIG. 6D shows a non-limiting example of a nutritional profile for an embodiment of the edible chew that contain glycerol.

### Example 3

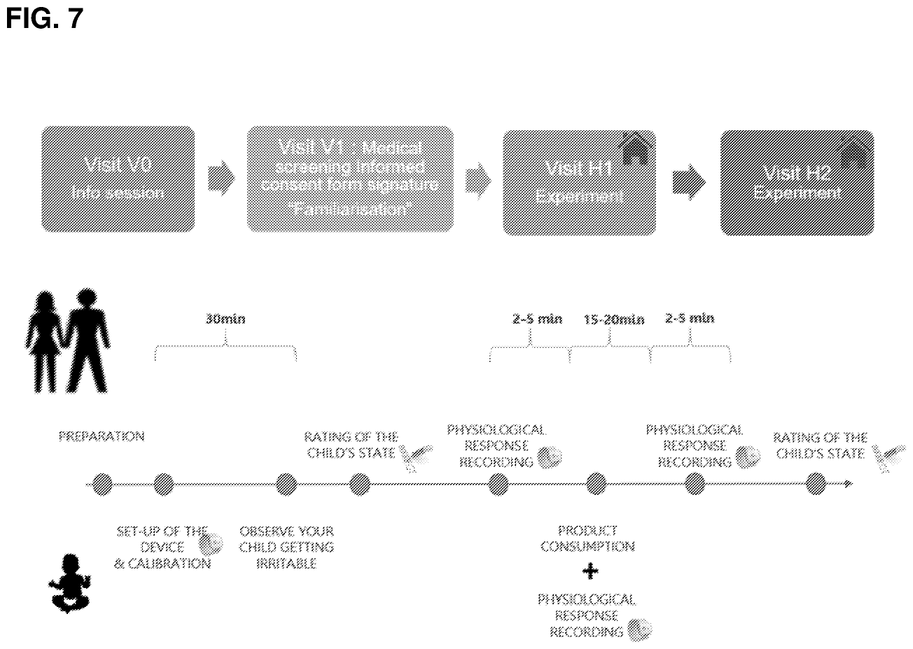

A study involving six babies aged from 6 to 18 months old investigated the effect of the edible soothing teether disclosed herein, based on changes induced by the exposure to the teether in mood and physiological state (FIG. 7). This was a home-based study and parents had to perform the measurements twice, on two different days. The measurements comprised parent-rated mood scores of their child (before and after exposure to the teether) and physiological recordings of the baby's heart rate using a wearable device (before, during and after exposure to the teether).

Parental mood ratings were obtained from 100-mm visual analog scales anchored from “not at all” to “extremely.” Heart rate was taken as an objective measure of the baby affective state based on the circumplex model of emotion originally developed by Russell (1980) composed of a two-dimensional circular space, containing arousal (stimulation-deactivation) and valence (positive-negative) dimensions. According to this model, a decrease in heart rate can translate into a positive state of contentment/calmness and an increase in heart rate into a positive state of excitement/pleasure (Kovacs and Bories 2010; Van Praag et al. 2017).

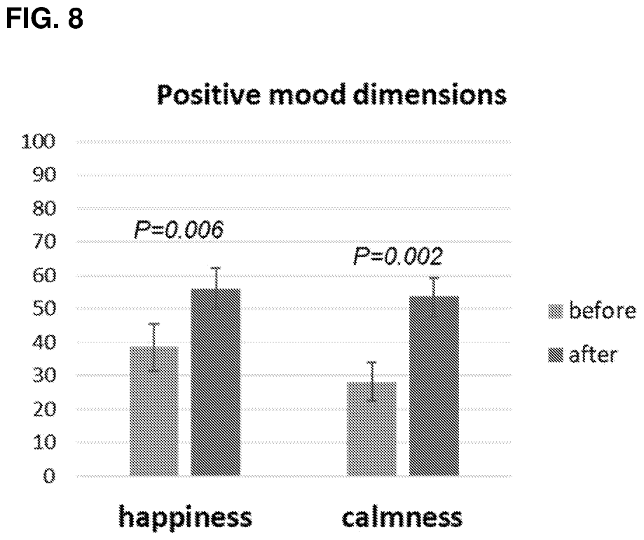

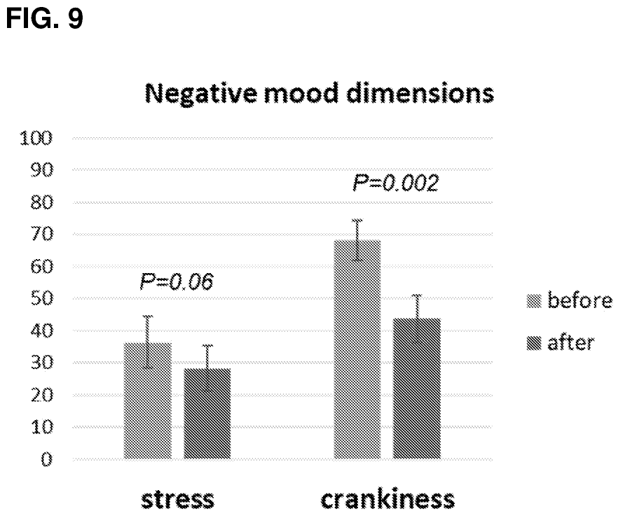

The parental rated mood score from 6 babies, including 5 babies with repeated measures are represented on FIGS. 8 and 9. General results show changes from before to after exposure to the edible teether, with increased positive mood scores and decreased negative mood scores. The results support the soothability benefit of the edible teether, in that babies were perceived by their parents as significantly more calm and less cranky, as well as marginally less stressed than before exposure to the teether.

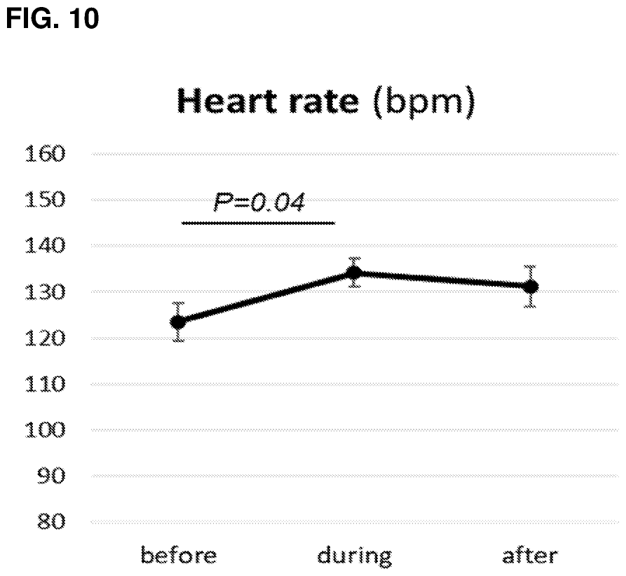

The dynamic changes in the babies' heart rate measured in 5 babies, including 4 babies with 2 repeated measures, are shown in FIG. 10. Results show a significant increase in heart rate during the exposure to the teether relative to before exposure. Based on the circumplex model of emotion (Russell 1980), such a change in heart rate can translate into a positive stimulation response supporting a pleasure benefit induced by the edible teether during its consumption. The pleasure benefit is further supported by normative heart rate data (Fleming et al. 2011) and by the parental perception of increased happiness (FIG. 8).

As described by Fleming et al. (2011), the median heart rate in infants under one year of age is between 127 beats/minute and 145 beats/minute. The results illustrated in FIG. 10 are consistent with the literature. The heart rate value of 123.5 beats/minutes at the “before” time-point could be considered as a value of resting state of the baby. The heart rate increases from 123 beats/minutes to 134 beats/minute to stabilize at 131 beats/minutes at the end of the experiment. This increase coincides with the mechanical action of chewing by the baby. In condition of calming effect, a decrease of heart rate value is expected through time, which is not represented in FIG. 10. However, combining mood rating scores with physiological measurement values shows an increase of heart rate while parents rated an increase of positive mood scores and a decrease of negative mood scores. This increase of babies heart rate could therefore be linked to a positive stimulation of infant's arousal during product consumption.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. An edible chew formulated for oral consumption by a human child, the edible chew comprising an expanded pre-gelatinized cereal flour matrix and a plasticizer:
wherein the human child is an infant as young as six months old or a toddler, and the edible chew is sized for the human child to hold the edible chew and place the edible chew in their mouth;
the edible chew take more than five (5) minutes to dissolve; and
the edible chew has a texture adapted for at least one effect during the oral consumption of the edible chew by the human child selected from the group consisting of (i) massaging gums of the human child, (ii) alleviating teething pain of the human child using elasticity of the edible chew, (iii) not adhering to gums of the human child, (iv) dissolving in the mouth of the child without breaking into pieces, and (v) helping the human child practice chewing.

2. The edible chew of claim 1, wherein the edible chew has at least one flavor selected from the group consisting of a cereal, a fruit, a vegetable, an herb and a spice.

3. The edible chew of claim 1, wherein the edible chew has a moisture content of 12-14 wt. % and a water activity not greater than 0.6.

4. The edible chew of claim 1, wherein the edible chew has at least one characteristic selected from the group consisting of an outer diameter of about 15 mm to about 20 mm, a length of about 50 mm to about 100 mm, and a weight of about 10 g to about 15 g.

5. The edible chew of claim 1, further comprising at least one active ingredient.

6. The edible chew of claim 5, wherein the at least one active ingredient is selected from the group consisting of a vitamin, a mineral, chamomile, lavender, a sugar alcohol other than glycerol, a cooling sensate, a micronutrient, a prebiotic, a probiotic, a protein, an amino acid, a nutraceutical, an essential oil, a homeopathic ingredient, a pharmaceutical compound, and mixtures thereof.

7. The edible chew of claim 1, wherein the edible chew is formulated to be at ambient temperature for the oral consumption by the human child.

8. The edible chew of claim 1, wherein the edible chew is formulated to be chilled or frozen for the oral consumption by the human child.

9. The edible chew of claim 1, wherein the edible chew has a bright color and/or a plurality of different colors.

10. The edible chew of claim 1, wherein the edible chew is one or more of organic, gluten-free or nut-free.

11. The edible chew of claim 1, wherein the edible chew comprises a potential food allergen in an amount effective for early tolerance introduction in the human child.

12. The edible chew of claim 1, wherein the hollow center is configured to reduce a risk of the human child choking on the edible chew during the oral consumption by the child.

13. The edible chew of claim 12, further comprising at least one filling within a hollow center.

14. The edible chew of claim 1, further comprising a holder configured to attach to the edible chew.

15. A method of (i) providing teething support to a human child who is teething and/or (ii) soothing, calming and/or delivering an active ingredient to a human child, the method comprising administering the edible chew formulated for oral consumption by a human child, the edible chew comprising an expanded pre-gelatinized cereal flour matrix and a plasticizer: wherein the human child is an infant as young as six months old or a toddler, and the edible chew is sized for the human child to hold the edible chew and place the edible chew in their mouth; the edible chew take more than five (5) minutes to dissolve; and the edible chew has a texture adapted for at least one effect during the oral consumption of the edible chew by the human child selected from the group consisting of (i) massaging gums of the human child, (ii) alleviating teething pain of the human child using elasticity of the edible chew, (iii) not adhering to gums of the human child, (iv) dissolving in the mouth of the child without breaking into pieces, and (v) helping the human child practice chewing.

16. The method of claim 15, wherein the edible chew is administered while having a temperature at or less than ambient temperature.

17. The method of claim 15, wherein the edible chew is administered while frozen.

18. A method of making an edible chew formulated for consumption by a human child, the method comprising:
forming a dry mix comprising a pre-gelatinized cereal flour;
metering the dry mix into an extruder comprising an extrusion die;
adding a plasticizer to the dry mix to form a dough;
promoting nucleation of the dough by subjecting the dough to a combination of shear and a temperature in a section of the extruder adjacent to the extrusion die, to form a heated and sheared dough; and
directing the heated and sheared dough through an opening of the extrusion die.

19. (canceled)

20. The method of claim 18, wherein the temperature in the section of the extruder adjacent to the extrusion die is at least 120° C.

21. The method of claim 18, wherein the opening of the extrusion die is at least partially defined by a mandrel that forms a hollow center in the edible chew.
